(19) United States
(12) Patent Application Publication
(10) Pub. No.: US 2017/0319461 A1
(43) Pub. Date: Nov. 9, 2017

(54) PHOTO-CURING NAIL GEL COMPOSITION

(71) Applicant: Prior Company Limited, Taipei City (TW)

(72) Inventors: I-Ling Chen, New Taipei City (TW); Chia-Le Lee, Taipei City (TW)

(73) Assignee: Prior Company Limited, Taipei City (GB)

(21) Appl. No.: 15/218,076

(22) Filed: Jul. 24, 2016

(30) Foreign Application Priority Data
May 9, 2016 (TW) 105114274

(51) Int. Cl. A61K 8/87; A61K 8/04; A61Q 3/02; A61K 8/46; A61K 8/73

(52) U.S. Cl. CPC A61K 8/87; A61K 8/46; A61K 8/731; A61Q 3/02; A61K 8/042; A61K 2800/42; A61K 2800/81; A61K 2800/95; A61K 2800/594

(57) ABSTRACT

A photo-curing nail gel composition is provided, including a polyurethane (meth)acrylate resin (resin elongation rate: 200% to 700%), a diluent, a photo initiator, a surface curing accelerating agent and a filler. The diluent includes a monofunctional monomer, a difunctional monomer and a trifunctional monomer. The photo initiator performs the photo-initiation reaction of the composition. The surface curing accelerating agent includes a tertiary amine acrylic resin or a thiol resin. A content of the polyurethane (meth)acrylate resin is 50 wt % to 80 wt %, contents of the monofunctional monomer, the difunctional monomer and the trifunctional monomer are respectively 1 wt % to 30 wt %, a content of the photo initiator is 1 wt % to 10 wt %, a content of the surface curing accelerating agent is 1 wt % to 10 wt % and a content of the filler is 1 wt % to 10 wt %, based on a total weight of the photo-curing nail gel composition.

### CROSS-REFERENCE TO RELATED APPLICATION

This application claims the priority benefit of Taiwan application serial no. 105114274, filed on May 9, 2016. The entirety of the above-mentioned patent application is hereby incorporated by reference herein and made a part of this specification.

### BACKGROUND OF THE INVENTION

### 1. Field of the Invention

The present invention generally relates to a photo-curing nail gel composition, in particular, to a photo-curing nail gel composition having properties of adhesion, beautification and scratch resistance.

### 2. Description of Related Art

In recent years, consumer's manicure requirements of nails coloring or decoration increase, and gel polish manicure using photo-curing nail gel has become one of the most important and commonly used technologies in modern nail polish market.

In traditional gel polish manicure technology, the photo-curing nail gel is usually a three-step gel including a base gel, a coloring gel and a top-coating gel, so the gel coating steps are complicated. More specifically, the nail gel is adhered to the nail by the base gel, the coloring gel has coloring and beautification effects, and the top-coating gel is the uppermost protection layer having the effects of preventing coloring gel scratching and enhancing brightness. However, as for the nail gel removal of the aforementioned three-step nail gel, it is required to apply the removal reagent with cotton pads and cover the nail with foil paper for a while, so as to lower the adhesion between the nail gel and the nail. In addition, it is also required to grind off the nail gel left on the nail during nail gel removal process. Therefore, it might cause damage to the nail surface when grinding off the nail gel, which is harmful to the nail and costs manual labor and time.

Based on the above, how to simplify the complicated steps of traditional photo-curing nail gel and simultaneously lower the possibility of nail damage in nail gel removal process is an issue that a person skilled in the art seeks to resolve.

### SUMMARY OF THE INVENTION

The invention provides a photo-curing nail gel composition, which is a one-step gel having properties of adhesion, beautification and scratch resistance, and it can be removed by stripping procedure.

The invention provides a photo-curing nail gel composition including a polyurethane (meth)acrylate resin with resin elongation rate of 200% to 700%, a diluent, a photo initiator, a surface curing accelerating agent and a filler. The diluent includes a monofunctional monomer, a difunctional monomer and a trifunctional monomer. The photo initiator performs the photo-initiation reaction of the composition. The surface curing accelerating agent includes a tertiary amine acrylic resin or a thiol resin. A content of the polyurethane (meth)acrylate resin is 50 wt % to 80 wt %, contents of the monofunctional monomer, the difunctional monomer and the trifunctional monomer are respectively 1 wt % to 30 wt %, a content of the photo initiator is 1 wt % to 10 wt %, a content of the surface curing accelerating agent is 1 wt % to 10 wt % and a content of the filler is 1 wt % to 10 wt %, based on a total weight of the photo-curing nail gel composition.

In an embodiment of the invention, the photo-curing nail gel composition includes a colorant, wherein a content of the colorant is 0.2 wt % to 3 wt %, based on a total weight of the photo-curing nail gel composition.

In an embodiment of the invention, the monofunctional monomer includes hydroxyethyl acrylate (HEA), hydroxyethyl methacrylate (HEMA), hydroxypropyl methacrylate (HPMA), acrylic acid (AA), methyl acrylic acid (MAA), isobornyl acrylate (IBOA) or isobornyl methacrylate (IBOMA).

In an embodiment of the invention, the difunctional monomer includes dipropylene glycol diacrylate (DPGDA), tripropylene glycol diacrylate (TPGDA), propoxylated neopentyl glycol diacrylate (NPG2PODA), 1,6-hexanediol diacrylate (HDDA), ethylene glycol dimethacrylate (EGDMA) or 2-hydroxylethyl methacrylate phosphate (HEMAP).

In an embodiment of the invention, the trifunctional monomer includes ethoxylated trimethylolpropane triacrylate (TMP3EOTA, TMP9EOTA and TMP15EOTA), propoxylated trimethylolpropane triacrylate (TMP3POTA) or propoxylated glyceryl triacrylate (G3POTA).

In an embodiment of the invention, the photo initiator includes diphenyl (2,4,6-trimethylbenzoyl)-phosphine oxide (TPO), ethyl(2,4,6-trimethylbenzoyl) phenylphosphinate (TPO-L), isopropyl thioxanthone (ITX) or camphorquinone (CQ).

In an embodiment of the invention, an absorption wavelength of the photo initiator is 320 nm to 400 nm.

In an embodiment of the invention, the filler includes methyl cellulose, carboxymethyl cellulose, nitrocellulose or acetyl cellulose.

In an embodiment of the invention, the photo-curing nail gel composition is cured under irradiation with a light source having a wavelength of 320 nm to 400 nm for 30 seconds.

Based on the above, the photo-curing nail gel composition of the invention is a one-step gel having properties of adhesion, beautification and scratch resistance. In other words, the photo-curing nail gel composition of the invention combines the property of each gel layer in the traditional three-step nail gel, so as to achieve the purpose of adhesion, beautification and scratch resistance by single nail gel. Therefore, the complicated nail coating steps of traditional three-step nail gel can be simplified, so as to achieve the purpose of saving manual labor and time. Besides, photo-curing nail gel composition of the invention can be removed by stripping procedure, so it is able to remove the nail gel easily without the removal reagent. As a result, it not only simplifies the traditional nail gel removal process which costs manual labor and time, but also lowers the possibility of nail damage during nail gel removal process.

The concepts and advantages of the present invention will become more apparent in view of the following embodiments and accompanying detailed information.

### DESCRIPTION OF THE EMBODIMENTS

Reference will now be made in detail to the present preferred embodiments of the invention. However, these embodiments are illustrative examples, and the invention is not limited thereto.

It should be mentioned that in the specification and claims of the invention, “polyurethane (meth)acrylate resin” represents “polyurethane acrylate resin” and/or “polyurethane methacrylate resin”.

The invention provides a photo-curing nail gel composition including a polyurethane (meth)acrylate resin, a diluent, a photo initiator, a surface curing accelerating agent, a filler and a colorant. The mentioned components will be described in detail below.

### <Polyurethane (Meth)Acrylate Resin>

A resin elongation rate of the polyurethane (meth)acrylate resin of the invention is 200% to 700%, which is the main toughness source of the colloid. Based on a total weight of the photo-curing nail gel composition, a content of the polyurethane (meth)acrylate resin is 50 wt % to 80 wt %, and preferably 50 wt % to 60 wt %. More specifically, the polyurethane (meth)acrylate resin of the invention may be obtained from commercially available products, such as: 6148-J75 produced by Eternal Materials Co., Ltd, CN966-J75 produced by Sartomer Co., DM553, DM5433H and DM5232 produced by Double Bond Chemical Inc., Co., Ltd. However, the invention is not limited thereto, and other commercially available products may also be used according to the actual operation condition.

### <Diluent>

The diluent of the invention may include a monofunctional monomer, a difunctional monomer and a trifunctional monomer, which is mainly used to adjust formula viscosity and increase colloid crosslinking, so as to enhance curing rate, toughness and nail surface adhesion.

The monofunctional monomer may include hydroxyethyl acrylate (HEA), hydroxyethyl methacrylate (HEMA), hydroxypropyl methacrylate (HPMA), acrylic acid (AA), methyl acrylic acid (MAA), isobornyl acrylate (IBOA) or isobornyl methacrylate (IBOMA).

The difunctional monomer may include dipropylene glycol diacrylate (DPGDA), tripropylene glycol diacrylate (TPGDA), propoxylated neopentyl glycol diacrylate (NPG2PODA), 1,6-hexanediol diacrylate (HDDA), ethylene glycol dimethacrylate (EGDMA) or hydroxylethyl methacrylate phosphate (HEMAP).

The trifunctional monomer may include ethoxylated trimethylolpropane triacrylate (TMP3EOTA, TMP9EOTA and TMP15EOTA), propoxylated trimethylolpropane triacrylate (TMP3POTA) or propoxylated glyceryl triacrylate (G3POTA).

Based on the total weight of the photo-curing nail gel composition, contents of the monofunctional monomer, the difunctional monomer and the trifunctional monomer are respectively 1 wt % to 30 wt %. More specifically, when the contents of the difunctional monomer and the trifunctional monomer are respectively 1 wt % to 5 wt %, suitable reactivity and crosslinking can be achieved. When the content of the monofunctional monomer is 5 wt % to 10 wt % and the content of the difunctional monomer is 10 wt % to 15 wt %, the adhesion with nail can be enhanced. Besides, a total content of the monofunctional monomer, the difunctional monomer and the trifunctional monomer is less than 40 wt %.

However, if the contents of the difunctional monomer and the trifunctional monomer are higher than the upper limit of the aforementioned range, the reaction shrinkage rate may be too high. If the contents of the monofunctional monomer and the difunctional monomer are higher than the upper limit of the aforementioned range, it may be difficult to remove the gel by stripping procedure. If the contents of the monofunctional monomer and the difunctional monomer are lower than the upper limit of the aforementioned range, the adhesion may be worse. In addition, if the total content of the monofunctional monomer, the difunctional monomer and the trifunctional monomer is higher than 40 wt %, the curing effect is worse. Therefore, it is unable for the composition to cure and dry under irradiation with a light source having a wavelength of 320 nm to 400 nm for 30 seconds, and it is unable to remove the gel by stripping procedure.

### <Photo Initiator>

The photo initiator of the invention performs a photo-initiation reaction of the photo-curing nail gel composition, and an absorption wavelength of the photo initiator is 320 nm to 400 nm. More specifically, the photo initiator may include diphenyl (2,4,6-trimethylbenzoyl)-phosphine oxide (TPO), ethyl(2,4,6-trimethylbenzoyl) phenylphosphinate (TPO-L), isopropyl thioxanthone (ITX) or camphorquinone (CQ). the photo initiator of the invention may be obtained from commercially available products, such as Irgacure® TPO, Irgacure® TPO-L, Irgacure®907, Irgacure®819 and Irgacure®369 produced by BASF Co. Based on the total weight of the photo-curing nail gel composition, the content of the photo initiator is 1 wt % to 10 wt %.

### <Surface Curing Accelerating Agent>

The surface curing accelerating agent of the invention may include tertiary amine acrylic resin or thiol resin, and it is able to reduce the oxygen inhibition effect in the acrylic resin curing system, so as to further increase the colloid surface curing effect. Based on the total weight of the photo-curing nail gel composition, a content of the surface curing accelerating agent is 1 wt % to 10 wt %. More specifically, when the content of the surface curing accelerating agent is 1 wt % to 5 wt % and the content of the photo initiator is 1 wt % to 10 wt %, a better surface curing effect can be achieved.

The surface curing accelerating agent of the invention may include pentaerythritoltetrakis(3-mercaptobutyrate), trimethylolpropanetris(3-mercaptopropionate) or pentaerythritoltetrakis(3-mercaptopropionate). Besides, the surface curing accelerating agent of the invention may be obtained from commercially available products, such as: KarenzMT PE1 produced by Showa Denko Electronics Co., Ltd., 641, 6410, 6411, 6417 and 6430 produced by Eternal Materials Co., Ltd, DM L234 produced by Double Bond Chemical Inc., Co., Ltd, TMMP and PEMP produced by SC Organic Chemical Co., Ltd., ADDITOL™ LED 01 produced by Cytec Industries Inc. However, the invention is not limited thereto, and other commercially available products may also be used according to the actual operation condition.

### <Filler>

The filler of the invention may include methyl cellulose, carboxymethyl cellulose, nitrocellulose or acetyl cellulose, wherein an esterification degree of acetyl cellulose is higher than 50%. Based on the total weight of the photo-curing nail gel composition, a content of the filler is 1 wt % to 10 wt %. More specifically, when the content of the filler is 2 wt % to 5 wt %, it is helpful to enhance the dispersing effect of pigment and surface curing effect.

### <Colorant>

Based on the total weight of the photo-curing nail gel composition, a content of the colorant is 0.2 wt % to 3 wt %.

The following experimental examples will be used to describe the photo-curing nail gel composition of the invention. However, the following experimental examples are not intended to limit the invention.

### EXPERIMENTAL EXAMPLE

The following experimental examples are used to prove that the photo-curing nail gel composition of the invention has properties of adhesion, beautification and scratch resistance, and it can be removed by stripping procedure.

### Preparation of Photo-Curing Nail Gel Composition

The photo-curing nail gel compositions of Experimental Examples 1 to 4 and Comparative Examples a to f are prepared, wherein the content of each component are shown in the following Table 1 and Table 2. Experimental Examples 1 to 4 are photo-curing nail gel compositions of the invention.

In Table 2, the polyurethane (meth)acrylate resins used in Comparative Example e and f are respectively 6113 and 6127 produced by Eternal Materials Co., Ltd.

### Evaluation 1: Curing and Drying Properties Evaluation After Light Irradiation

The photo-curing nail gel compositions of Experimental Examples 1 to 4 and Comparative Examples a to f and commercially available products A to D are applied on nails, and then irradiated with a LED light source having a wavelength of 320 nm to 400 nm for 30 seconds. Afterwards, curing and drying properties are evaluated. The evaluation results are shown in the following Table 1, Table 2 and Table 3, and the evaluation standards are as follows:
Excellent ◯: Nearly no gel is left (<5%), which can be removed by alcohol wiping.
Tolerable Δ: More gel is left (>5%), which can be removed by alcohol wiping.
Poor x: It is totally unable to cure the composition.

### Evaluation 2: Stripping Property Evaluation

After the photo-curing nail gel compositions of Experimental Examples 1 to 4 and Comparative Examples a to f and commercially available products A to D applied on nails are cured, try to strip off the cured composition or the cured commercially available products with fingers and observe whether they can be stripped off, so as to evaluate the stripping property. The evaluation results are shown in the following Table 1, Table 2 and Table 3, and the evaluation standards are as follows:
Excellent ◯: It can be stripped off as a whole without broken fragments.
Tolerable Δ: It can be stripped off, but it is also separated into small fragments.
Poor x: It is totally unable to strip off.

TABLE 1

| | | Experimental | Experimental | Experimental | Experimental |
|---|---|---|---|---|---|
| Component | Type | Example 1 | Example 2 | Example 3 | Example 4 |
| Polyurethane | DM5433H | 65 wt % | | | |
| (meth)acrylate | DM553 | | 65 wt % | | |
| resin | CN966-J75 | | | 65 wt % | |
| | DM5232 | | | | 65 wt % |
| Filler | Acetyl cellulose | 4 wt % | 4 wt % | 4 wt % | 4 wt % |
| Diluent | HEA | 12 wt % | 12 wt % | | |
| | (monofunctional | | | | |
| | monomer) | | | | |
| | HEMA | | | 12 wt % | 12 wt % |
| | (monofunctional | | | | |
| | monomer) | | | | |
| | TMP3EOTA | 4 wt % | | 4 wt % | 4 wt % |
| | (trifunctional | | | | |
| | monomer) | | | | |
| | G3POTA | | 4 wt % | | |
| | (trifunctional | | | | |
| | monomer) | | | | |
| | HDDA | 5 wt % | | | |
| | (difimctional | | | | |
| | monomer) | | | | |
| | EGDMA | | 5 wt % | | |
| | (difunctional | | | | |
| | monomer) | | | | |
| | NPG2PODA | | | 5 wt % | |
| | (difunctional | | | | |
| | monomer) | | | | |
| | DPGDA | | | | 5 wt % |
| | (difunctional | | | | |
| | monomer) | | | | |
| | HEMAP | 4 wt % | | | 4 wt % |
| | (monofunctional | | | | |
| | monomer) | | | | |
| | AA | | 4 wt % | 4 wt % | |
| | (monofunctional | | | | |
| | monomer) | | | | |
| Surface curing | Karenz MT PE1 | 3 wt % | 3 wt % | 3 wt % | 3 wt % |
| accelerating | (thiol resin) | | | | |
| agent | | | | | |
| Photo initiator | Irgacure ® | 2 wt % | 2 wt % | 2 wt % | 2 wt % |
| | TPO-L | | | | |
| Colorant | | 1 wt % | 1 wt % | 1 wt % | 1 wt % |
| Total | | 100 wt % | 100 wt % | 100 wt % | 100 wt % |
| Evaluation | Evaluation 1: | ∘ | ∘ | ∘ | ∘ |
| results | curing and | | | | |
| | drying properties | | | | |
| | evaluation after | | | | |
| | light irradiation | | | | |
| | Evaluation 2: | ∘ | ∘ | ∘ | ∘ |
| | stripping property | | | | |
| | evaluation | | | | |
| | Gel type | One-step gel | One-step gel | One-step gel | One-step gel |

TABLE 2

| | | Comparative | Comparative | Comparative | Comparative | Comparative | Comparative |
|---|---|---|---|---|---|---|---|
| Component | Type | Example a | Example b | Example c | Example d | Example e | Example f |
| polyurethane | DM5433H | 45 wt % | | | | | |
| (meth)acrylate | DM553 | | 45 wt % | | | | |
| resin | CN966-J75 | | | 45 wt % | | | |
| | DM5232 | | | | 45 wt % | | |
| | 6113 | | | | | 60 wt % | |
| | 6127 | | | | | | 60 wt % |
| Filler | Acetyl cellulose | 3.5% | 3.5% | 3.5% | 3.5% | 5 wt % | 5 wt % |
| Diluent | HEA | 13.5 wt % | 13.5 wt % | | | 13.5 wt % | 15 wt % |
| | (monofunctional | | | | | | |
| | monomer) | | | | | | |
| | IBOA | | | 13.5 wt % | | | |
| | (monofunctional | | | | | | |
| | monomer) | | | | | | |
| | HEMA | | | | 13.5 wt % | | |
| | (monofunctional | | | | | | |
| | monomer) | | | | | | |
| | TMP3EOTA | 8 wt % | | 8 wt % | 8 wt % | 8 wt % | |
| | (trifunctional | | | | | | |
| | monomer) | | | | | | |
| | G3POTA | | 8 wt % | | | | 8 wt % |
| | (trifunctional | | | | | | |
| | monomer) | | | | | | |
| | HDDA | 12 wt % | | | | | 3 wt % |
| | (difunctional | | | | | | |
| | monomer) | | | | | | |
| | EGDMA | | 12 wt % | | | | |
| | (difunctional | | | | | | |
| | monomer) | | | | | | |
| | NPG2PODA | | | 12 wt % | | 3 wt % | |
| | (difunctional | | | | | | |
| | monomer) | | | | | | |
| | DPGDA | | | | 12 wt % | | |
| | (difunctional | | | | | | |
| | monomer) | | | | | | |
| | HEMAP | 10 wt % | | | 10 wt % | 1 wt % | |
| | (monofunctional | | | | | | |
| | monomer) | | | | | | |
| | AA | | 10 wt % | 10 wt % | | | 1 wt % |
| | (monofunctional | | | | | | |
| | monomer) | | | | | | |
| Surface curing | Karenz MT PE1 | 5 wt % | 5 wt % | 5 wt % | 5 wt % | 5 wt % | 5 wt % |
| accelerating | (thiol resin) | | | | | | |
| agent | | | | | | | |
| Photo initiator | Irgacure ® TPO-L | 2 wt % | 2 wt % | 2 wt % | 2 wt % | 2 wt % | 2 wt % |
| Colorant | | 1 wt % | 1 wt % | 1 wt % | 1 wt % | 1 wt % | 1 wt % |
| Total | | 100 wt % | 100 wt % | 100 wt % | 100 wt % | 100 wt % | 100 wt % |
| Evaluation | Evaluation 1: | Δ | x | x | Δ | Δ | Δ |
| results | curing and | | | | | | |
| | drying properties | | | | | | |
| | evaluation after | | | | | | |
| | light irradiation | | | | | | |
| | Evaluation 2: | Δ | x | x | Δ | x | x |
| | stripping property | | | | | | |
| | evaluation | | | | | | |
| | Gel type | One-step gel | One-step gel | One-step gel | One-step gel | One-step gel | One-step gel |

TABLE 3

| | | Commercially | Commercially | Commercially | Commercially |
|---|---|---|---|---|---|
| | | available | available | available | available |
| | | product A | product B | product C | product D |
| Evaluation | Evaluation 1 | Δ | x | x | Δ |
| results | curing and drying | | | | |
| | properties | | | | |
| | evaluation after | | | | |
| | light irradiation | | | | |
| | Evaluation 2: | x | x | x | x |
| | stripping property | | | | |
| | evaluation | | | | |
| | Gel type | One-step gel | One-step gel | Three-step gel | Three -step gel |

As shown in Table 1, Experimental Examples 1 to 4 are photo-curing nail gel composition of the invention, which is a one-step gel having properties of adhesion, beautification and scratch resistance. Besides, the photo-curing nail gel compositions of Experimental Examples 1 to 4 are totally cured under irradiation with a light source (LED) having a wavelength of 320 nm to 400 nm for 30 seconds with fast-curing property. As for adhesion, the photo-curing nail gel compositions of Experimental Examples 1 to 4 do not detach from the nail in about 7 days. In addition, the photo-curing nail gel compositions of Experimental Examples 1 to 4 can be easily stripped off as a whole by fingers without broken fragments after cured by irradiation, so the nail gel can be removed without removal reagent. Therefore, the stripping property of the photo-curing nail gel compositions of Experimental Examples 1 to 4 is good.

On the contrary, in the photo-curing nail gel compositions of Comparative Examples a to f, the content of each component is out of the range of content of each component in the photo-curing nail gel composition of the invention. Therefore, the curing and drying properties of Comparative Examples a to f under irradiation with a light source (LED) having a wavelength of 320 nm to 400 nm for 30 seconds are poor, and the stripping property is also poor. More specifically, the total content of the monofunctional monomer, the difunctional monomer, and the trifunctional monomer in Comparative Example b and c is higher than 40 wt %, so Comparative Example b and c can't be cured under irradiation with a light source (LED) having a wavelength of 320 nm to 400 nm for 30 seconds with poor curing property, and Comparative Example b and c can't be removed by stripping procedure.

Based on the above, the photo-curing nail gel composition of the invention is a one-step gel having properties of adhesion, beautification and scratch resistance, so as to achieve the purpose of adhesion, beautification and scratch resistance by single nail gel. Therefore, the complicated nail coating steps of traditional three-step nail gel can be simplified, so as to achieve the purpose of saving manual labor and time. As for the adhesion property, the adhesion time is about 7 days. Besides, the photo-curing nail gel composition of the invention is totally cured under irradiation with a light source having a wavelength of 320 nm to 400 nm for 30 seconds with fast-curing property. On the other hand, the photo-curing nail gel composition of the invention can be stripped off without broken fragments, so the nail gel can be easily removed without removal reagent. As a result, it not only simplifies the traditional nail gel removal process which costs manual labor and time, but also lowers the possibility of nail damage during nail gel removal process.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A photo-curing nail gel composition, comprising:
a polyurethane (meth)acrylate resin, wherein a resin elongation rate of the polyurethane (meth)acrylate resin is 200% to 700%;
a diluent, wherein the diluent includes a monofunctional monomer, a difunctional monomer and a trifunctional monomer;
a photo initiator, which performs a photo-initiation reaction of the photo-curing nail gel composition;
a surface curing accelerating agent, wherein the surface curing accelerating agent includes a tertiary amine acrylic resin or a thiol resin; and
a filler,
wherein a content of the polyurethane (meth)acrylate resin is 50 wt % to 80 wt %, contents of the monofunctional monomer, the difunctional monomer and the trifunctional monomer are respectively 1 wt % to 30 wt %, a content of the photo initiator is 1 wt % to 10 wt %, a content of the surface curing accelerating agent is 1 wt % to 10 wt % and a content of the filler is 1 wt % to 10 wt %, based on a total weight of the photo-curing nail gel composition.

2. The photo-curing nail gel composition of claim 1, further comprising a colorant, wherein a content of the colorant is 0.2 wt % to 3 wt %, based on a total weight of the photo-curing nail gel composition.

3. The photo-curing nail gel composition of claim 1, wherein the monofunctional monomer includes hydroxyethyl acrylate (HEA), hydroxyethyl methacrylate (HEMA), hydroxypropyl methacrylate (HPMA), acrylic acid (AA), methyl acrylic acid (MAA), isobornyl acrylate (IBOA) or isobornyl methacrylate (IBOMA).

4. The photo-curing nail gel composition of claim 1, wherein the difunctional monomer includes dipropylene glycol diacrylate (DPGDA), tripropylene glycol diacrylate (TPGDA), propoxylated neopentyl glycol diacrylate (NPG2PODA), 1,6-hexanediol diacrylate (HDDA), ethylene glycol dimethacrylate (EGDMA) or 2-hydroxylethyl methacrylate phosphate (HEMAP).

5. The photo-curing nail gel composition of claim 1, wherein the trifunctional monomer includes ethoxylated trimethylolpropane triacrylate (TMP3EOTA, TMP9EOTA and TMP15EOTA), propoxylated trimethylolpropane triacrylate (TMP3POTA) or propoxylated glyceryl triacrylate (G3POTA).

6. The photo-curing nail gel composition of claim 1, wherein the photo initiator includes diphenyl (2,4,6-trimethylbenzoyl)-phosphine oxide (TPO), ethyl(2,4,6-trimethylbenzoyl) phenylphosphinate (TPO-L), isopropyl thioxanthone (ITX) or camphorquinone (CQ).

7. The photo-curing nail gel composition of claim 1, wherein an absorption wavelength of the photo initiator is 320 nm to 400 nm.

8. The photo-curing nail gel composition of claim 1, wherein the filler includes methyl cellulose, carboxymethyl cellulose, nitrocellulose or acetyl cellulose.

9. The photo-curing nail gel composition of claim 1, wherein the photo-curing nail gel composition is cured under irradiation with a light source having a wavelength of 320 nm to 400 nm for 30 seconds.
